(19) United States
(12) Patent Application Publication
(10) Pub. No.: US 2005/0177160 A1
(43) Pub. Date: Aug. 11, 2005

(54) Dynamic cervical plate

(76) Inventors: Bret O. Baynham, Jupiter, FL (US); G. Clay Baynham, Jupiter, FL (US); Matthew G. Baynham, Jupiter, FL (US); David R. Campbell, Jupiter, FL (US)

Correspondence Address: MCHALE & SLAVIN, P.A., 2855 PGA BLVD, PALM BEACH GARDENS, FL, 33410, US

(21) Appl. No.: 10/776,369

(22) Filed: Feb. 10, 2004

(51) Int. Cl. A61B 17/56

(52) U.S. Cl. 606/69

(57) ABSTRACT

A dynamic cervical plate has a ratchet and pawl mechanism that allows the cervical plate to post operatively shorten the length of the plate and maintain compression between adjacent vertebrae. The plate has an elongated shaft with teeth on one surface and a groove along each longitudinal edge. A lateral bar is attached on one end of the shaft. The bar has screw holes for connecting with the head of a spinal screw. Another lateral bar is slidably engaged in the longitudinal grooves along the shaft and has a spring clip acting as a pawl with the teeth on the shaft. The sliding bar has screw holes on each side of the shaft. The clip is configured to span the screw holes to prevent screws from backing out of the holes.

### BACKGROUND OF THE INVENTION

1. Field of the invention

This invention relates to the field of orthopedic surgery and, particularly, to the area of spinal implants for stabilizing the spatial relationship of vertebrae. The device is designed for use in the cervical region of the spine though one skilled in the art may use the device in other regions of the spine and other skeletal fixations.

2. Description of the Prior Art

Spinal plates are well known in the orthopedic art for fixing bones or bone fragments in a pre-selected spatial orientation. The plates are usually attached to the bones or bone fragments by screws designed to make a secure and long lasting connection not affected by the loads caused by normal activities of the host. Gertzbein et al, U.S. Pat. No. 5,620,443, teaches an adjustable cervical connector composed of dual rods spanning the distance between adjacent vertebrae. The rods carry at least two slidable transverse connectors which are attached to the vertebrae by spikes and pedicle screws thereby fixing the relationship of the bones. The connectors are immobilized on the rods by clamps.

Richelsoph, U.S. Pat. No. 6,017,345, teaches a spinal plate spanning the distance between adjacent verrtebrae. The plate has screw holes in each end. The pedicle screws are inserted through the holes and allow for some movement.

Shih et al, U.S. Pat. No. 6,136,002, teaches a similar device to that of Gertzbein with the clamps screwed onto the elongated rods.

Published Patent Application US 2003/0060828 A1 to Michelson teaches a cervical plate with at least two plate elements slidably connected together and fixed by a set screw. The contacting surfaces of the plate elements are formed with ratcheting to provide added security.

In all these prior art devices, the plate must be held in the selected position while the securing set screws or other fasteners are put in place and the final assembly is completed.

What is needed in the art is a dynamic cervical plate that may be adjusted to length, locked in place to provide compression, and will automatically shorten its length to maintain compression.

### SUMMARY OF THE PRESENT INVENTION

Therefore, it is an objective of this invention to provide a cervical plate with an elongated shaft adapted to span the intervertebral space and having at least two screw receivers spaced along the length of the plate. The screw receivers each have screw holes for accepting the heads of pedicle screws.

Another objective of this invention is to provide a locking mechanism that is manually operated simultaneously with the positioning of the screw receivers along the plate to provide compression across the intervertebral space.

A further objective of this invention is to provide the locking mechanism with a retainer extending over the screw holes to prevent back out of the screws.

Yet another objective of this invention is to provide a guide rail on the plate shaft cooperating with the screw receivers to permit sliding connection between the screw receivers and the plate shaft.

Still another objective of this invention is to provide a ratchet mechanism on the shaft and screw receivers to permit post operative one-way movement shortening the distance between the screw receivers and maintaining compression across the intervertebral space.

### SHORT DESCRIPTION OF THE DRAWINGS

FIG. 1 is a perspective of the cervical plate and screw receivers of this invention;

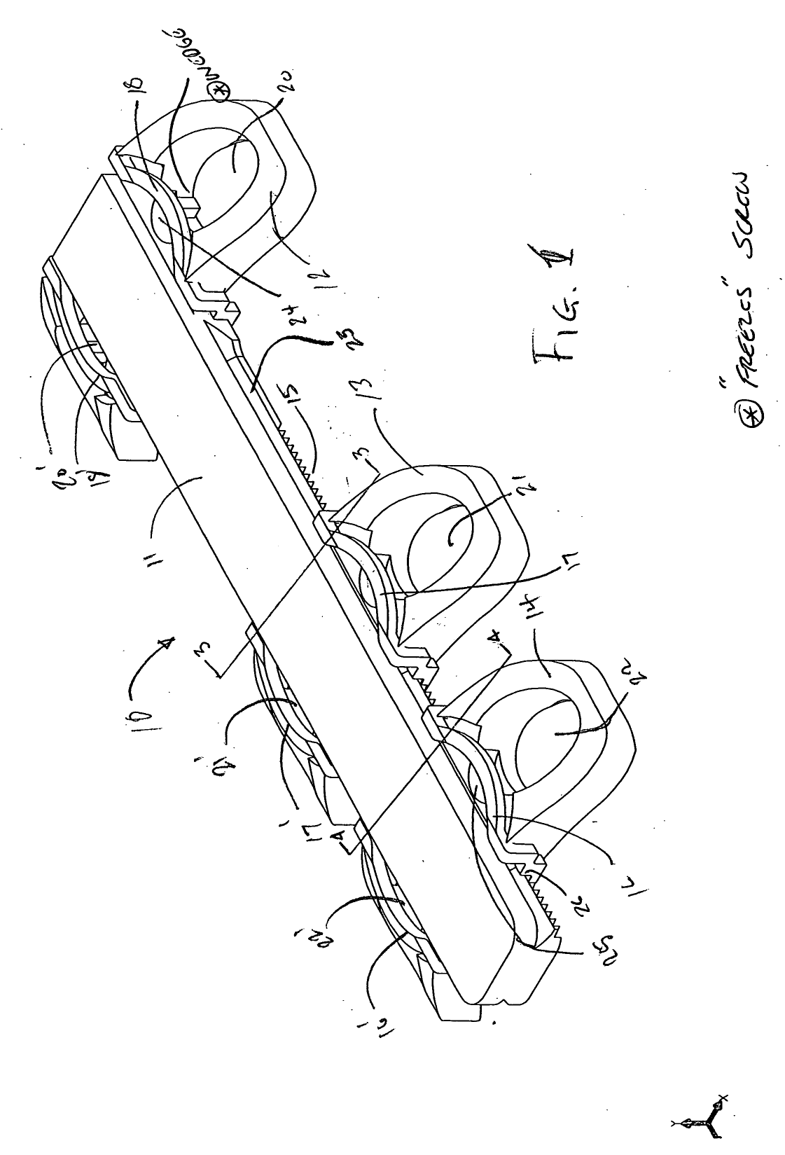

FIG. 2 is a bottom plan view of the cervical plate and screw receivers;

FIG. 3 is a cross section of the cervical plate, along line 3-3 of FIG. 1, with the clip unseated; and

FIG. 4 is a cross section of the cervical plate, along line 4-4 of FIG. 1, with the clip seated in the ratchet.

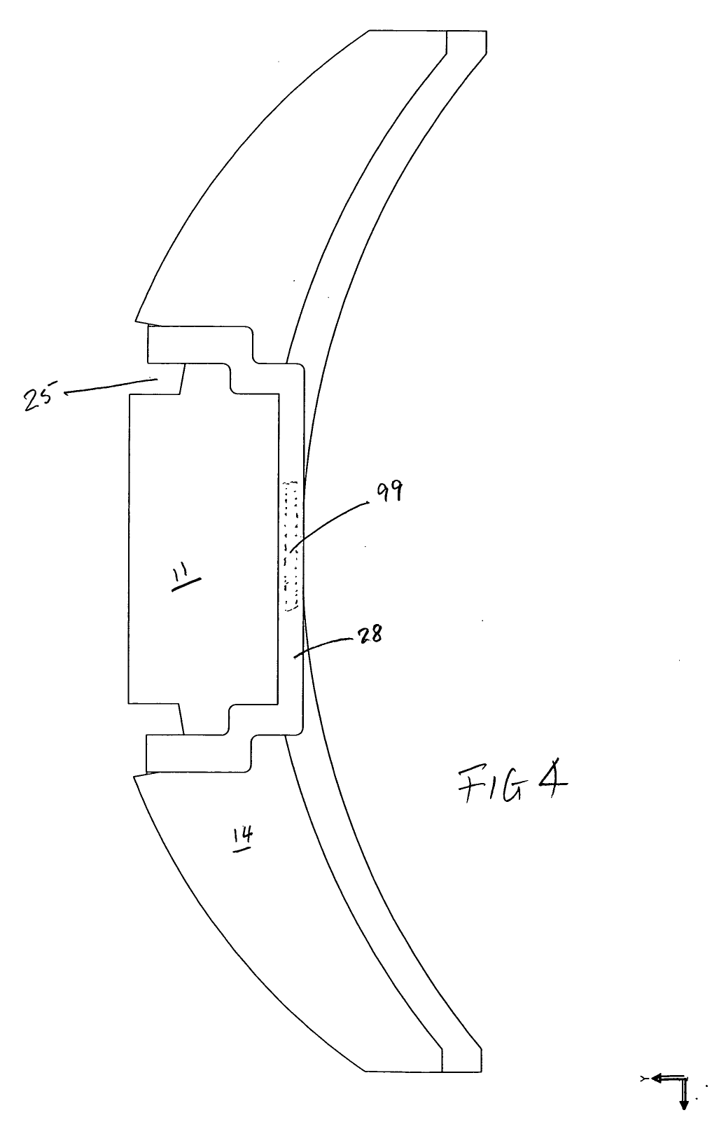

### DETAILED DESCRIPTION OF THE INVENTION

The cervical plate 10 has an elongated flat shaft that is made in different lengths but must be of a length to span, at least, the distance between two vertebrae. The plate has a lateral bar 12 fixed to one end and a free end. The bar has an countersunk apertures 20, 20′ on each side of the plate for capturing the head of pedicle screws. Permanently mounted to the plate is a clip having ears 18, 18′. The clip is resilient and extends under the plate parallel but outside the periphery of the bar then rises vertically to the top of the plate and extends across the pedicle screw apertures 20, 20′. The portion that extends across the countersunk apertures 20, 20′ are the ears 18, 18′ for retaining the pedicle screws to prevent back-out. The retainer is resilient enough to allow flexing while the heads of the pedicle screws are seated in the aperture then is released on top of the screw heads. In one embodiment, the ears 18, 18′ have wedges 50 which engage the edges of the screw heads as the screws are tightened to further lock the screws in place.

The bottom of the shaft has a row of teeth15 formed across the longitudinal axis of the plate. The teeth are angled to form a ratchet allowing one-way movement of a bar from the free end toward the lateral bar at one end of the plate. In some instances, the teeth may be cut normal to the shaft. Along each longitudinal side of the shaft is a groove 23, 23′ extending from the free end toward the lateral bar.

Slidably attached to the free end of the shaft is at least one movable bar 13 but two are preferred. The second bar 14 is of the same construction as the bar 13. Bar 13 and bar 14 have the same structure therefore, reference to elements of one bar is the same as the other.

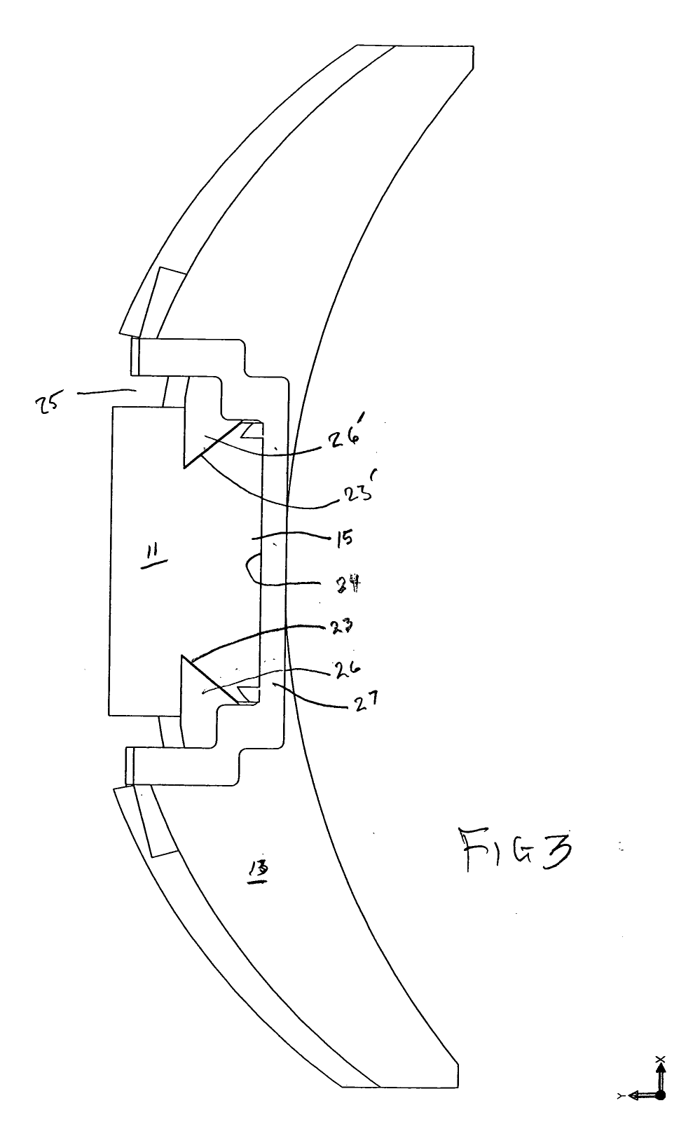

The slidable bar 13 has a distal surface which engages the vertebrae and is convexly curved to closely fit the curvature of the vertebrae. The bar has an aperture 21, 21′ near each end with a depression 25 therebetween The depression is approximately the same depth as the thickness of the shaft to provide a low profile to the assembled cervical plate. The opposite edges of the depression have shoulders 26, 26′, shown in FIG. 3, that slide within the longitudinal grove 23, 23′ in the plate. This provides a close association between the surface of the bar depression and the ratchet teeth of the plate.

Attached to bar 13 is a clip having a retainer 17, 17′. The clip has an elongated hollow body with an oval shape. The sides of the oval follow the edges of the depression so that the retainers 17, 17′ are on the proximal surface of the bar. At least one side of the clips is welded 99 or otherwise permanently attached to the respective side of the bars. The rounded ends of the oval form the screw retainers. The pawl portion 27, 28 of the clips extend across the shaft engaging the teeth 15 to form the ratchet. The pawls are formed by a raised flange 24, shown in FIG. 3.

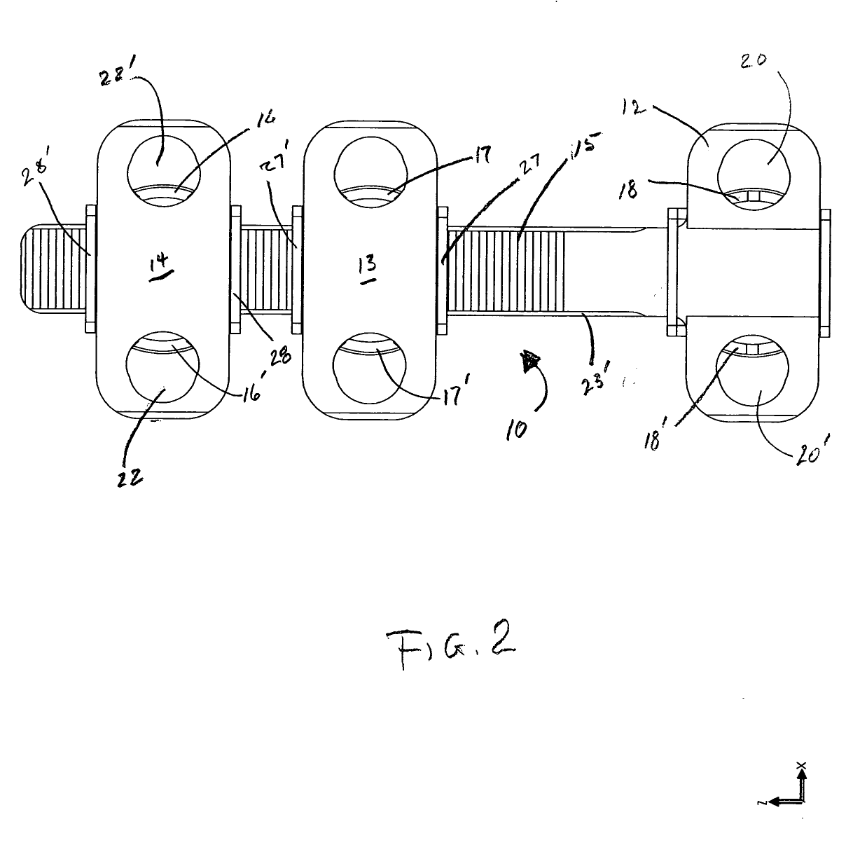

In the preferred embodiment, the clips 16 and 17 have a flange that extends above the surfaces of the bars to engage the teeth 15 of the ratchet on the shaft. Of course, the clips may have pawls 27, 27′ and 28, 28′ on both sides of the bar, shown in FIG. 2. By flexing the clip with an instrument, the flange 24 can be disengaged from the ratchet teeth 15, as shown in FIG. 3, for initial adjustment.

In operation, the vertebrae are manipulated into the desired position and grafting material placed as required to compensate for removal of bone and/or disk material. The plate is placed on the spine and adjusted to provide some compression on the site to assist in the grafting of the spine. As the bars are slid along the shaft, the shoulders of the bars and the grooves on the shaft maintain a close fit between the pawls and the teeth on the shaft requiring the pawls to be deflected by the teeth. Once the bars are in the desired location and the flanges seated in the teeth, the ratchet prevents retrograde movement of the bars away from the head. The pedicle screws are driven into the spine. As the screw heads engage the apertures the retainers are flexed to permit the screw heads to seat in the apertures and released to block back-out.

It is well known that as the site heals and the adjacent vertebrae begin to graft together and as a result of the forces of gravity, there is some reduction in the span between the vertebrae. As this occurs the dynamic cervical plate can accommodate the reduction and maintain some compression because the shaft will move in the bars resulting in the clips moving from one ratchet tooth to the next automatically shortening the intervertebral distance.

The second bar may be added to the free end of the plate to add stability to the compressed site and to reduce and equalize the pressure. Of course, the pawls may be omitted, and the plate may move in both directions within the bars.

A number of embodiments of the present invention have been described. Nevertheless, it will be understood that various modifications may be made without departing from the spirit and scope of the invention. Accordingly, it is to be understood that the invention is not to be limited by the specific illustrated embodiment but only by the scope of the appended claims.

1. A dynamic spinal plate for stabilizing adjacent vertebrae comprising and elongated shaft with a proximal surface and a distal surface, said elongated shaft having a first bar near one end adapted for connection with a vertebra, said shaft having an opposite end, at least a second bar movably attached near said opposite end, said first and second bars each having a screw hole adapted to seat a screw head, a clip attached to said second bar, said clip having a retainer spanning said screw hole.

2. A dynamic spinal plate of claim 1 wherein said first bar is fixed to said shaft, said first bar extending transverse to said elongated shaft, screw holes in said first bar on each side of said elongated shaft, a clip attached to said first bar, said clip having a retainer spanning each of said screw holes.

3. A dynamic spinal plate of claim 2 wherein said second bar extends transverse to said elongated shaft, screw holes in said second bar on each side of said elongated shaft, said clip attached to said second bar having a retainer spanning each of said screw holes.

4. A dynamic spinal plate of claim 1 wherein said elongated shaft has a longitudinal groove along each side, said second bar including a central depression with shoulders on each side, each of said shoulders engaging said longitudinal groove along each side of said shaft for slidable movement of said second bar along said shaft with said central depression in close contact with said elongated shaft.

5. A dynamic spinal plate of claim 2 wherein said elongated shaft has a longitudinal groove along each side, said second bar including a central depression with shoulders on each side, each of said shoulders engaging said longitudinal groove along each side of said shaft for slidable movement of said second bar along said shaft with said central depression in close contact with said elongated shaft.

6. A dynamic spinal plate of claim 3 wherein said elongated shaft has a longitudinal groove along each side, said second bar including a central depression with shoulders on each side, each of said shoulders engaging said longitudinal groove along each side of said shaft for slidable movement of said second bar along said shaft with said central depression in close contact with said elongated shaft.

7. A dynamic spinal plate of claim 3 wherein a third bar is slidably attached near said opposite end of said elongated shaft, said third bar extends transverse to said elongated shaft, screw holes in said third bar on each side of said elongated shaft, a clip fixed to said third bar having a retainer spanning each of said screw holes.

8. A dynamic spinal plate of claim 7 wherein said elongated shaft has a longitudinal groove along each side, said second bar and said third bar each including a central depression with shoulders on each side, each of said shoulders engaging said longitudinal groove along each side of said shaft for slidable movement of said third bar along said shaft with said central depression in close contact with said elongated shaft.

9. A dynamic spinal plate of claim 1 wherein said shaft has transverse teeth formed in said distal surface along the length thereof, said clip having a pawl portion seated between adjacent teeth.

10. A dynamic spinal plate of claim 2 wherein said shaft has transverse teeth formed in said distal surface along the length thereof, said clip attached to said second bar having a pawl portion seated between adjacent teeth.

11. A dynamic spinal plate of claim 3 wherein said shaft has transverse teeth formed in said distal surface along the length thereof, said clip attached to said second bar having a pawl portion seated between adjacent teeth.

12. A dynamic spinal plate of claim 4 wherein said shaft has transverse teeth formed in said distal surface along the length thereof, said clip attached to said second bar having a pawl portion seated between adjacent teeth.

13. A dynamic spinal plate of claim 7 wherein said shaft has transverse teeth formed in said distal surface along the length thereof, each said clip attached to said second and said third bar having a pawl portion seated between adjacent teeth.

14. A dynamic spinal plate of claim 9 wherein said transverse teeth are angled toward said opposite end whereby said shaft may advance through said second bar shortening the distance between said first bar and said second bar, said pawl portion and said transverse teeth preventing lengthening said distance.

15. A dynamic spinal plate of claim 14 wherein said first bar is fixed to said shaft, said first bar extending transverse to said elongated shaft, screw holes in said first bar on each side of said elongated shaft, a clip attached to said first bar, said clip having a retainer spanning each of said screw holes, said second bar extends transverse to said elongated shaft, screw holes in said second bar on each side of said elongated shaft, a clip attached to said second bar having a retainer spanning each of said screw holes, said elongated shaft having a longitudinal groove along each side, said second bar including a central depression with shoulders on each side, each of said shoulders engaging said longitudinal groove along each side of said shaft for slidable movement of said second bar along said shaft with said central depression in close contact with said elongated shaft.

16. A dynamic spinal plate of claim 15 wherein a third bar is slidably attached near said opposite end of said elongated shaft, said third bar extends transverse to said elongated shaft, screw holes in said third bar on each side of said elongated shaft, a clip fixed to said third bar having a retainer spanning each of said screw holes, said clip fixed to said third bar including a pawl portion, whereby said shaft may advance through said third bar shortening the distance between said first bar, said second bar, and said third bar, said pawl portion of said clip fixed to said third bar and said transverse teeth preventing lengthening said distance.

17. A dynamic spinal plate for stabilizing adjacent vertebrae comprising an elongated shaft with a proximal surface and a distal surface, said shaft having one bar fixed at one end adapted for connection with a vertebra, said bar extending laterally normal to said elongated shaft, screw holes in said one bar on each side of said elongated shaft, said shaft having an opposite free end, said shaft having a set of transverse teeth formed on said distal surface, at least a second bar movably attached near said free end of said plate, said second bar adapted for connection with an adjacent vertebra, said second bar extending laterally normal to said elongated shaft, screw holes in said second bar on each side of said elongated shaft, said second bar including an attached clip, said clip having a flange engaging said teeth and securing said second bar along the length of said plate thereby maintaining the space between said one bar and said second bar.

18. A dynamic spinal plate of claim 17 wherein said elongated shaft has a longitudinal groove along each side, said second bar including a central depression with shoulders on each side, each of said shoulders engaging said longitudinal groove along each side of said shaft for slidable movement of said second bar along said shaft with said central depression in close contact with said elongated shaft.

19. A dynamic spinal plate of claim 18 wherein said transverse teeth are angled toward said opposite end whereby said shaft may advance through said second bar shortening the distance between said first bar and said second bar, said pawl portion and said transverse teeth preventing lengthening said distance.

20. A dynamic spinal plate of claim 19 including a third bar movably attached near said free end, said third bar having a central depression with shoulders on each side, each of said shoulders engaging said longitudinal groove along each side of said shaft for slidable movement of said third bar along said shaft with said central depression in close contact with said elongated shaft, said third bar including an attached clip, said clip having a flange engaging said teeth and securing said third bar along the length of said plate thereby maintaining the space between said one bar and said second bar.
